(19) United States
(12) Patent Application Publication
(10) Pub. No.: US 2002/0106388 A1
(43) Pub. Date: Aug. 8, 2002

(54) Formulation of flavones and isoflavones for treatment of cellulite

(76) Inventor: Peter T. Pugliese, Berneville, PA (US)

Correspondence Address: ARTHUR R. EGLINGTON, 113 Cross Creek Dr. R.D. # 5., Pottsville, PA, 17901, US

(21) Appl. No.: 09/989,019

(22) Filed: Nov. 21, 2001

Related U.S. Application Data
(60) Provisional application No. 60/250,397, filed on Nov. 24, 2000.

(51) Int. Cl. A61K 31/7048; A61K 31/522; A61K 7/00; A61K 35/78

(52) U.S. Cl. 424/401; 424/725; 514/27; 514/263.31; 514/456

(57) ABSTRACT

Disclosed is a treatment protocol and formulation for the cosmetic condition known as cellulite. The active ingredients of the several formulation include isoflavones, like genistein, a hydroxyflavone, like quercetin, a xanthine derivative, like theophylline, a carnitine, and a plant extract like coleus forskohlii. In the emusion form, standard emulsifiers, emollients, and preservatives are utilized. In the microencapsulated form, the slurry also includes a carrier and preservative, which facilitates a slurry that is used to impregnate body garments, like pantyhose, for extended application to the user's skin.

### CROSS REFERENCE TO OTHER FILINGS

This is an examinable application submitted for an official PTO filing receipt under 35 U.S. Code Section 111(a). This is a continuation-in-part application of my copending provisional application of Nov. 24, 2000, accorded U.S. Ser. No. 60/250,997, same title.

### FIELD OF THE INVENTION

This patent application relates to a method and composition for the control and treatment of a cosmetic condition known as cellulite. Treatment is directed at controlling the breakdown of collagen and reducing the fat mass to a smaller volume.

### BACKGROUND OF THE INVENTION

Cellulite is a term imported from the French to describe a condition of the buttocks and upper thighs characterized by an unattractive, undulating, irregular skin surface. The condition has been thought by previous investigators to originate from abnormal fatty deposits that collected under the skin. Many attempts have been made to define cellulite but no adequate explanation has been forthcoming. While fat is not the major etiological factor in cellulite, it is an important component of the condition. ¹Kuiper, GJM, et all Interaction of Estrogenic Chemicals and Phytoestrogens with Estrogen Receptor β Endocrinology 139:4252-4263, 1998

It is recognized that phytoestrogens have an estrogenic effect on certain cells of the body and these effects relate to the binding capacity of phytoestrogens on specific estrogen receptor sites in the body.¹ Once the estrogen receptor is bound by estrogens it undergoes conformational changes allowing the receptor to interact with high affinity towards chromatin and to modulate the transcription of target genes. Originally only one estrogen receptor was believed to present on a cell but now it is known that at least two receptors and α receptor and β receptor are present.² It was learned later that some of the natural phytoestrogens have different binding affinities to the two receptors in that the binding to the β receptor was much higher than to the α receptor. Various phytoestrogens have a markedly different binding effects to either α or β receptor and the difference appears to be related to the presence or absence of one or two hydroxyl groups on the phytoestrogen molecule. ²Kuiper, G J M, et all Cloning of a novel receptor expression in rate prostate and ovary. Proc Natl Acad Sci 93:5925:5939, 1996

As a result, the phtyoestrogens have the capacity to act as either partial estrogen agonists, or antagonists depending on the expression of estrogen receptors subtypes in the cell, and the concentration of the phytoestrogen. Clinically, phytoestrogens may exert tissue specific effects. Phytoestrogens also exhibit both estrogen receptor dependent and independent effects that suggest additional mechanisms beyond receptor binding. Induced differentiation of cancer cells and inhibition of tyrosine kinase are two known effects.

Estrogens are known to stimulate fibroblasts to produce collangenase, a mechanism that is improtant in removing the endometrial decidua in the menstrual cycle. The stimulation of fibroblast to release collagenase is not specific to the endometrium so wide ranging effects are produced by this action of estrogen on fibroblast. The breakdown of collagen in the fatty tissues and loss of collagen in the joints results in weakening of these structures. Currently, it is unclear what specific effects are produced by which receptor.

Stored Lipids—the Source of Cellulite

Adipocytes serve as the major storage units for fat. Even though some cells actually contain droplets of lipid material, they are a minor source of stored energy. Where and why lipids are stored at certain sites remains a mystery, but much is known about some of the mechanism that control the deposition of fat at certain sites, such as the hips and thighs. With a high fat and high carbohydrate diet the lipid storage is initiated by insulin. Insulin inhibits the utilization of fat whenever glucose is available. When the body needs lipids, the triglycerides must be broken down again. This process is called lipolysis.

Cellulite has been treated by a variety of physical and chemical methods. These include body wraps using cloth and plastic materials with and without added herbal extracts, massage both by hand and machine, baths using salts and mud, exercise and diet. The use of agents that are capable of lipolytic stimulation, such as the xanthine derivatives, caffeine and theophylline, has shown some efficacy in published clinical trials. These treatments offer results which are at best temporary, and do not address the primary cause which is estrogen.

It is well established that estrogen helps prevent dry skin and wrinkles as well as offering protection against osteoporosis in postmenopausal women. Studies have shown that estrogen promotes the formation of collagen and elastin in skin and that falling estrogen levels are associated with skin laxity. Oral estrogen replacement remains controversial since there appears to be some risk of associated breast cancer increase as well as an increased incidence of heart disease. On the other hand, phytoestrogens have less affinity for breast and uterine tissue than conjugated estrogens.

Genistein, 5,7-Dihydroxy-3-(4-hydroxyphenyl)-4H-1-benzopyran-4-one; 4′,5,7-trihydroxyisoflavone; a phytoestrogen, has high affinity for bone and heart tissue and less affinity for breast and uterine tissues.³ ³Cassidy, A. Potential selectivity of dietary phytoestrogens and estrogens. Curr Opin Lipidol. 10:45-57.

Mixed reports relate the effects of estrogen on connective tissue as being either supportive of collagen and elastin increase, or as increasing the breakdown of these tissue components. Genistein is reported to inhibit tyrosine kinase and thereby promote the formation of collagen and elastin and at the same time to inhibit by an estrogen independent method, the action of matrix metallproteases which break down collagen and elastin.^{4,5} ⁴ Shao, Z et all Genistein inhibits both constitutive and EGF-stimulated invasion in Er-negative human breast carcinoma cell lines. Anticancer Res 18:1435-1440, 1998. ⁵Yoon, H K, et al Differential effects of two protein tyrosine kinase inhibitors, tryphotsin and genistein, on human bone cell proliferation as compared with differentiation. Calcif. Tissue Int 63:243-249, 1998

Genistein is known to occupy estrogen cell receptors and block the action of estrogen. Cellulite is a condition seen only in women and is characterized by the formation of fatty deposits in specific sites of the body, namely in the thighs and the buttocks. These ft reserves are allocated by the body as an energy depot specifically for nourishment of the fetus during lean times. Experience shows that no amount of dieting or exercise can reduce the area of the thighs and buttocks in women with normal estrogen levels. Even in female body builders who have high testosterone levels, there remains a certain amount of cellulite. Ultrasound studies of cellulite indicate a general weakening of the collagen connective tissue and an increase in fatty deposits that extend into the dermis. These deposits push against the dermis and create an undulating surface effect that is characteristic of cellulite.

The mechanism underlying cellulite appears to be as follows:

1. At the time of puberty the estrogen level rises, causing a stimulation of fibroblasts to produce collagenase.

2. Collagenase specifically attacks the superficial fascia, producing a weakening of the collagen fibers and allowing an area of broken fascia to appear. Fatty tissue herniates through this fascia and pushes upwards into the dermis.

3. The dermis is also weakened by the action of collagenase on the collagen fibers in the dermis, and thus offers no resistance to the upward movement of the fatty tissue. The net result is the undulating appears of the outer skin as the fat pushes upwards.

4. The fatty tissue produces more estrogen, resulting in greater fibroblast activity and more collagenase being produced. As a result, cellulite is a progressive, self-perpetuating condition.

### SUMMARY OF THE INVENTION

According to the present invention, a treatment protocol for the cosmetic condition in females, commonly known as cellulite, involves novel utilization of the following inter-related physiological precepts. Estrogen can be inhibited by blocking estrogen receptors (ER) located on the fibroblast cells with a selected isoflavone, like genistein. Collagen fibers form a tough fibrous tissue termed the fascia, which hold fatty tissues in discrete compartments. Collagenase breaks down collagen fibers. The action of collagenase can be blocked at the level of collagen by employing proanthocyanidins. The production of estrogen from testosterone can be blocked by another isoflavone like genistein. This isoflavone also blocks the initiation of preadipocytes by estrogen, to form fully-differentiated adipocytes, thus inhibiting the expansion of fat mass. Fatty tissue may also be reduced by stimulating the formation of cyclic adenosine triphosphate (cAMP), which cause the activation of lipase on fatty tissue, with coleus forskohlii serving as an effective stimulator of cAMP. The xanthine, theophylline acetate, inhibits the enzymatic action of phosphodiesterase which destroys cAMP, thus prolonging the lipase stimulating action of cAMP. Finally, a cofactor like acetyl carnitine is employed to increase the metabolism of the released free fatty acids by the mitochondria.

By the use of formulations compounded and applied according to the present invention, the positive clinical effects that may be observed are: the total estrogen level is reduced in the fatty tissue; collagen destruction by collagenase is reduced, or eliminated, in the skin areas to which the formulation is being regularly applied; the lipolytic process (lipase action) is stimulated so as to increase fat metabolism and to reduce fat mass; the skin is restructured to prevent further herniation of fatty tissue; and the dermis is strengthened as the collagen is no longer being destroyed.

This invention describes the cosmetic use of phytoestrogens, like genistein, quercetin and/or fisetin to treat cellulite. Our studies indicate that cellulite is confined to an area extending from the level of the pelvic brim to approximately four inches above the thighs. Published studies report that fatty deposits in this area are not easily metabolized by internal lipolytic pathways. In addition, preadipocytes in this area are converted to adipocytes whenever the body has either a need for more storage of fatty tissue, as in increased food intake, or when more space is available in this area. Dissolution of collagen produces more relative space in the superficial and deep fatty layer which promotes the conversion of preadipocytes to adipocytes.

### DETAILED DESCRIPTION OF A PREFERRED EMBODIMENT

An effective treatment program has been devised for cellulite that consists of the following ingredients that may be applied topically, either in a cream, lotion, or gel. The ingredients may be affixed to a garment, such as panty hose or a stocking, in micro-encapsulated form; or they may be applied as a material patch, or plaster, either in a gel form or cloth supported form.

Our invention teaches that cellulites may be treated effectively by using biochemical agents that are derived from plants which are capable of selectively blocking the action of endogenous estrogen and stimulating lipolysis. Specifically, the use of genistein blocks the action of estradiol on the estrogen receptor in the fibroblast of the connective tissue. More specifically, this receptor appears to be the estrogen receptor β. By blocking this receptor, the fibroblasts in the area of the cellulite will not be stimulated to produce more collagenase and elastase. This action allows the body to affect a repair of the damaged connective tissue. At the same time, genistein is capable of stimulating lipolysis in the area of cellulite.⁶ Genistein is known to inhibit tyrosine kinase which promotes the formation of the matrix metalloprotein collagenase from fibroblasts.⁷ ⁶Kuppusamy, U R, and Das, N P. Effects of flavanoids on cyclic AMP phosphodiesterase and lipid mobilizations in rat adipocytes. Biochem Pharmacol 44:1307-1315, 1992. ⁷Akiyama, T et al Genistein a specific inhibitor of tyrosine kinase. J. biol Chem 262:5592-5, 1987.

The combined effect of genistein and quercetin, 2-(3,4-Dihydroxyphenyl)-3,5,7-trihydroxy-4H-1-benzopyran-4-one; 3,3′,4′,5,7-penta hydroxyflavone; diadzein, 4′7-dihydroxyisoflavone; or fisten 2-(3,4-Dihydroxyphenyl)-3,7-dihydroxy-4H-1-benzopyran-4-one; 3,3′,4′,7-tetrahydroxyflavone; 5-desoxyquercetin; on cellulite, is to reduce the destruction of collagen tissue and promote the lipolysis of fatty tissue. This combination is an effective of treating cellulite as well as treating aging skin since both conditions are associated with collagen destruction. Genistein is also known to stimulate collagen formation, while quercetin is a powerful antioxidant as well as a lipolytic agent.

The active ingredients consist of the following:

1. An isoflavone, such as genistein, diadzein, or other isoflavones, that have an affinity for estrogen receptors on fibroblasts. The competitive action blocks the stimulation of estrogen to produce collagenase.

2. A hydroxyflavone, either quercetin or fisetin combined with the isoflavone;

3. A xanthine derivative, such as theophylline acetate, caffeine, theobromine, all of which blocks the action of the enzyme phosphodiesterase on cyclic AMP, to allow the process of lipolysis to continue by breaking down triglycerides, and the forming of fatty acids.

4. Acetyl carnitine that is required to move the fatty acids into the mitochondria for metabolism.

5. Coleus forskohlii, a plant extract that stimulates the action of cyclic AMP, so that it produces more active lipase for fatty acid generation by lipolysis.

6. A complex of other bioflavanoids [11^{th} Ed. Merck, #1241], capillary protectants, such as the oligomeric proanthrocyadins [11^{th} Ed. Merck, #2694] (collagenase inhibitors), ranging from 0.1 to 2.0 weight percent.

According to the invention, a non-toxic formulation comprises:

(a) an isoflavone, preferably genistein, 5,7-Dihydroxy-3-(4-Hydroxyphenyl)-4H-1-benzopyran-4-one [Merck Index, Monograph, #4281-11th edition], or another substituted benzopyran-4-one, like isoflavone [11^{th} Ed. Merck, #5057], ranging from 0.1 to 2.0 weight percent;

(b) a hydroxyflavone, like diadzein [11^{th} Ed. Merck #2805], preferably quercetin [11^{th} Ed. Merck, #8044], or fistetin (Merck #4026) ranging from 0.1 to 2.0 weight percent;

(c) theophylline acetate, [Merckh Idex #9212-11^{th} Ed.] ranging from 0.1 to 2.0 weight percent;

(d) a 3-hydroxy-4-(trimethylammonio)butanoate, like L-carnitine acetate, C₇H₁₅NO₃ [11^{th} Ed. Merck, #1856] which moves the fatty acids into the mitochondria, ranging from 0.1 to 3.0 weight percent; and,

(e) a plant extract termed coleus forskohlii (a cofactor), which stimulates the action of cAMP, ranging from 0.5 to 2.5 weight percent.

Also taught is a method of treating cellulite in which the disclosed ingredients may be in an anhydrous vehicle, such as alcohol, a vegetable, or mineral, or synthetic lipid material.

Further taught is a method of treating cellulite in which the ingredients may be in a gel formulation made from a natural gum, such as xanthan gum, or carrageenan, or a synthetic gelling agent, such as methoxycellulose, ethoxycellulose, or carboxycellulose, acyanoacrylate, polyvinylpyrolidine, or chitsan.

Selection of Active Ingredients

Ingredients for these formulations must be assayed by a gas chromatography mass spectrometer (GCMS). Suitable standards are employed to quantitate the ingredients. All ingredients must be at least 99.5% pure, that is, free of extraneous compounds.

### Formula #1 for Anti-cellulite Product (Emulsion Form)

| | Concentration, | w = water phase, |
|---|---|---|
| Ingredients | percent by wt | o = oil phase |
| Fisetin | .001 to 5.0 | o |
| Genistein | 0.1 to 2 | o |
| Theophylline acetate | .001 to 4.0 | w |
| Coleus forskohlii extract | .001 to 2.0 | w |
| L-carnitine | .001 to 2.0 | o |
| Oligomeric proanthyrocyandins | 0.1 to 2.0 | |
| Retinal (Vitamin A) | 0.05 to 2.0 | |
| Phenoxyethanol | 0.2 to 0.3 | Preservative |
| Glycerol monostearate | 3.0 to 8.0 | Emulsifier |
| Cetyl alcohol | 1.0 to 4.0 | Emollient |
| Isopropyl Palmitate | 2.0 to 4.0 | Emollient |
| Water qs | 100 | |

Preparation

Solubilize fisetin, genistein and carnitine in the ethanol. Place the water-soluble ingredients (theophylline and coleus forskohlii) in water phase and heat to 70 degrees C. Heat oil phase to 70 degrees and add to water phase. Stir, and when an emulsion forms, add ethanol-solubilized ingredients. Cool to 40 degrees C, and add preservatives.

To Treat Cellulite

Apply to upper legs and buttocks twice a day, in the morning and one hour before bedtime. Treatment should be continued for 60 days at this application level, and then be reduced to application one hour before bedtime only.

### Formula #2 Application by Microencapsulation

| Ingredients | Concentration, percent by wt | |
|---|---|---|
| Quercetin | .001 to 5.0 | |
| Genistein | .001 to 5.0 | |
| Theophylline acetate | .001 to 4.0 | |
| Coleus forskohlii extract | .001 to 2.0 | |
| L-carnitine | .001 to 1.5 | |
| Isopropyl palmitate | q.s. to 100% | Carrier |
| Phenoyxethanol | 1.0 to 4.0 | Preservative |

The above ingredients of Formula #2 are microencapsulated separately, and the microcapsules are mixed in an aqueous slurry in the proportion given above. The slurry is then placed in a large dipping vat and the pantyhose is dipped and stirred for two hours. The panty hose are then dried first by centrifugation, followed by hot air dryers.

### Formula #3 for Anti-cellulite Product (Emulsion Form)

| | Concentration, | w = water phase, |
|---|---|---|
| Ingredients | percent by wt | o = oil phase |
| Quercetin | .001 to 5.0 | o |
| Genistein | .001 to 5.0 | o |
| Theophylline acetate | .001 to 4.0 | w |
| Coleus forskohlii extract | .001 to 2.0 | w |
| Acetyl L-carnitine | .001 to 1.5 | o |
| Oligomeric proanthyrocyandins | 0.1 to 2.0 | |
| Glycerol monostearate | 3.0 to 8.0 | Emulsifier |
| Cetyl alcohol | 1.0 to 4.0 | Emollient |
| Isopropyl Palmitate | 2.0 to 4.0 | Emollient |
| Phenoxyethanol | 1.0 to 4.0 | Preservative |

The above-identified compounds are administered exactly as described for Formula #1, except quercetin in place of fisetin and acetyl carnitine and theophylline in place of their congeners.

Protocol for Treating Cellulite

The pantyhose are worn 8 hours a day, washed nightly, and changed to a new pair on the 4^{th} day. Once cellulite is improved, pantyhose may be worn only 2-3 times a week.

Testing of Candidate Product Efficacy

The following procedures are reported in many published articles for testing cellulite product efficacy. It is the method used in assessing the candidate products.

To determine the effectiveness of either the emulsion formula, or the pantyhose/microencapsulation preparation, the following parameters are measured:

2. Thigh diameter. The thigh diameter is measured with a cloth tape at a point six inches from the greater trochanter. Three measurements are taken and the average of all three measurements taken as the actual recorded reading.

3. Ultrasound measurements. The same anatomical site that is used to measure the thigh diameter is also used to make ultrasound scans of the epidermis/dermis to assess dermal density. The ultrasound unit is a 20 megahertz system, that will allow sound penetration to approximately 13 mm of the dermis and fat layers.

4. Photographic records. All subjects in the study are photographed using a digital camera. Four views are taken for each subject for each session, anterior, posterior and laterals. The area will cover the hips and two thirds of the upper legs, to assess skin continuity.

Procedure

Thirty subjects will be used for each formulation to be studied. All subjects will have the three parameters evaluated on day 1, on day 30, and day 60 of the study. The data is tabulated and recorded. The results are reported as percent decrease, or increase, of initial values obtained compared to the final values. For the emulsion formula, the subjects will use the product in the morning one hour before breakfast and again one hour before bedtime. For the pantyhose product, the subjects will wear the panty hose for eight hours a day, washing them every night. New panty hose will be issued and worn every fourth day of the study.

Interpretation of Observed Data

The tested product is considered to be effective based on the following criteria:

1. A reduction in thigh diameter of 1.0 centimeter, or more, over 60 days.

2. An increase in dermal density as measured by ultrasound.

3. A decrease in the amount of fat seen in the dermis and in the epidermis (the fat will appear as irregular echo free areas in the dermis and epidermis).

4. Observable changes on the photographs indicating a smoother appearance of the skin of the thighs and the buttocks.

5. A change in the appearance of the buttocks and thighs in that they are firmer and more rounded rather than amorphous in shape.

We claim:

1. An oil-in-water emulsion suited for topical application to the skin of a woman evincing the cosmetic condition called cellulite, having the active ingredients comprising:
(a) an oil-soluble hydroxyflavone selected from the group consisted of quercetin and fisetin;
(b) an oil-soluble isoflavone selected from the group consisting of genistein, diadzein, and biochanin-A;
(c) an oil-soluble amino substituted butanoate selected from the group consisting of DL-carnitine, L-carnitine, and carnitine acetate;
(d) a water-soluble xanthine selected from the group consisting of theophylline, theophylline salts, caffeine, and theobromine;
(e) a water-soluble plant extract coleus forskohlii;
(f) a solubizing agent for the oil-soluble ingredients; and,
(g) an emulsifying agent for mixing the oil phase with the aqueous phase.

2. The oil-in-water emulsion of claim 1 wherein quercetin is the hydroxyflavone present in the weight percent of the emulsion volume up to about 5%.

3. The oil-in-water emulsion of claim 1 wherein fisetin is the hydroxyflavone and is present in the weight volume up to about 5%.

4. The oil-in-water emulsion of claim 1 wherein the isoflavone is genistein and is present in the weight volume of up to about 5%.

5. The oil-in-water emulsion of claim 1 wherein the xanthine is theophylline acetate and is present in the weight volume of up to about 4%.

6. The oil-in-water emulsion of claim 1 wherein the coleus extract is present in the weight volume of up to about 2%.

7. The oil-in-water emulsion of claim 1 wherein the butanoate is L-acetyl carnitine and is present in the weight volume of up to about 1.5%.

8. The method of treating cellulitis in women wherein the application formulation comprises an oil-in-water emulsion comprising:
(a) an oil-soluble hydroxyflavone selected from the group consisted of quercetin and fifecetin;
(b) an oil-soluble isoflavone selected from the group consisting of genistein, diadzein, and biochanin-A;
(c) an oil-soluble amino substitute butanoate selected from the group consisting of DL-carnitine, L-carnitine, and carnitine acetate;
(d) a water-soluble xanthine selected from the group consisting of theophylline, theophylline salts, caffeine, and theopromine;
(e) a water-soluble plant extract coleus forskohlii;
(f) a solubizing agent for the oil-soluble ingredients; and,
(g) an emulsifying agent for mixing the oil phase with the aqueous phase.

9. The oil-in-water emulsion of claim 1 which has been converted to a gel consistency by the incorporation of a natural gum selected from xanthan gum, and carrageenan.

10. The oil-in-water emulsion of claim 1 which has been converted to a gel consistency by the incorporation of a gelling agent selected from one of methoxycellulose, ethoxycellulose, carboxycellulose, acyanacrylate, polyvinylpyrolidine, or chitsan.

11. The oil-in-water emulsion of claim 1 wherein the bioflavonoid oligomeric proanthrocyandins are included.
